(19) United States
(12) Patent Application Publication
(10) Pub. No.: US 2005/0255152 A1
(43) Pub. Date: Nov. 17, 2005

(54) System for enhanced targeted delivery

(75) Inventors: David A. Edwards, Boston, MA (US); Daniel R. Deaver, Franklin, MA (US); Thaddeus R.F. Fulford-Jones, Cambridge, MA (US); Jane N. Kucera, Acton, MA (US); Shang-You Tee, Allston, MA (US)

Correspondence Address: PATREA L. PABST;PABST PATENT GROUP LLP, 400 COLONY SQUARE, SUITE 1200, ATLANTA, GA, 30361, US

(73) Assignee: President and Fellows of Harvard College

(21) Appl. No.: 10/511,413

(22) PCT Filed: Apr. 17, 2003
(86) PCT No.: PCT/US03/12261

§ 371 Date: May 9, 2005

Related U.S. Application Data
(60) Provisional application No. 60/373,915, filed on Apr. 18, 2002.

(51) Int. Cl. A61K 38/22; A61K 9/127; A61K 9/14

(52) U.S. Cl. 424/450; 514/12; 424/489

(57) ABSTRACT

A method and compositions for targeted drug delivery have been developed. The compositions include a targeting molecules such as a hormone that specifically binds to a receptor on the surface of the targeted cells; a drug to be delivered, such as a toxin that will kill the targeted cells; and a nanoparticle, which contains on or within the nanoparticle, the drug to be delivered, as well as has attached thereto, the targeting molecule. Nanoparticles can consist of drug or drug associated with carrier, such as a controlled or sustained release materials like a poly(lactide-co-glycolide), a liposome or surfactant. The compositions are administered by injections in most cases, although compositions can be applied topically orally, nasally, vaginally, rectally, and ocularly. Compositions can also be administered to the pulmonary or respiratory system, most preferably in an aerosol.

### FIELD OF THE INVENTION

The present invention is generally in the field of targeted drug delivery, especially hormone targeted cytotoxic drugs in a microparticulate formulation, optionally in combination with a gel enhancing cell permeation. In one embodiment, the system consists of a simple and quick injection that causes immediate, permanent sterilization of an animal.

This application claims priority to U.S. Ser. No. 60/373,915 “System for Enhanced Targeted Delivery” Filed Apr. 18, 2002, by David A. Edwards.

### BACKGROUND OF THE INVENTION

Currently, the main method of spaying and neutering is surgical ovariectomies and orchiectomies. This method has a number of drawbacks. First, it is a time intensive and labor-intensive procedure, especially in the case of ovariectomies, which are understandably more invasive. Second, the surgical procedures are relatively costly. According to figures from the American Humane Society, the national average cost for cat castration is about $65 while for dogs the average is approximately $140. Lastly, many pet owners fail to get their pets fixed because of the violent nature of the surgical solution. Owners feel that their relationship with the pet will be adversely affected by the operation.

The American Veterinary Medical Association (AVMA) reports about 62 MM cats and 53 MM dogs nationwide with 77% of the cats and 50% of the dogs receiving surgical sterilization. Based on a 7-year average life cycle for cats and a 10-year average life cycle for dogs, approximately 10.53 MM companion animals a year require sterilization. The current cost of sterilization, and therefore the potential yearly total revenue as well as market size, is $443.3 MM for cats and $519.5 MM for dogs, or $962.7 MM total, according to the AVMA.

Four to six million healthy companion animals are euthanized every year according to Humane Society statistics at a cost to local governments throughout the US of $2 billion. This cost to local government is increased by populations of larger mammals, especially deer, which both spread disease as well as cause the insurance industry millions of dollars yearly due to injury to person and property.

Currently, the only method of pet sterilization is surgical. Single procedures range in cost from $50-$150. There is no single-injection sterilization product currently on the market. However, there does exist a drug called Leupron, which causes temporarily sterilization, and is used on humans to treat prostate and breast cancer. The drawbacks of Leupron are its cost, about $2,000 per injection, and the fact that it is only a temporary solution and thus must be readministered. Hormonal contraceptive treatments, involving injections, sprays, or lotions all temporarily remove the ability to reproduce, yet require repeated dosages. They currently are used primarily in older animals, and represent a very small fraction of the market.

It is often difficult to deliver compounds, such as proteins, peptides, genetic material, and other drugs and diagnostic compounds intracellularly because cell membranes resist the passage of these compounds. Various methods have been developed to administer agents intracellularly. For example, genetic material has been administered into cells in vivo, in vitro, and ex vivo using viral vectors, DNA/lipid complexes, and liposomes. While viral vectors are efficient, questions remain regarding the safety of a live vector and the development of an immune response following repeated administration. Lipid complexes and liposomes appear less effective at transfecting DNA into the nucleus of the cell and potentially may be destroyed by macrophages in vivo.

Considerable interest exists with respect to the subject of sterilization of animals. This is especially true of those concerned with veterinary medicine and animal husbandry, particularly as they relate to the subject of sterilization of domestic animals. Various methods have been developed over the years to accomplish sterilization. For example, with respect to male cattle, the most widely used procedure for eliminating problems of sexual or aggressive behavior is sterilization through surgical castration. This is done in various ways, e.g., crushing the spermatic cord, retaining the testes in the inguinal ring, or use of a rubber band, placed around the neck of the scrotum, to cause sloughing off of the scrotum and testes. However most of these “mechanical” castration methods have proven to be undesirable in one respect or another; for example they (1) are traumatic, (2) introduce the danger of anesthesia, (3) are apt to produce infection, and (4) require trained personnel. Moreover, all such mechanical castration methods result in complete abolition of the testes and this of course implies complete removal of the anabolic effects of any steroids which are produced by the testes and which act as stimuli to growth and protein deposition.

These drawbacks have caused consideration of various alternative sterilization techniques such as the use of chemical sterilization agents. However, the use of chemical sterilization agents has its own set of advantages and disadvantages. On the positive side, chemical sterilization eliminates the stress and danger associated with mechanical castration. Chemical sterilization also has the added advantage of allowing for retention of certain anabolic effects resulting from a continued presence of low levels of circulating testosterone. This is especially valuable in the case of animals raised for human consumption since circulating testosterone promotes growth, efficiency of feed conversion and protein deposition. Unfortunately, there are several disadvantages associated with chemical sterilization. For example chemical sterilization is often temporary rather than permanent; and it also sometimes produces extremely severe, and even fatal, side effects. Many of these chemical sterilization methods have been aimed at regulation of luteinizing hormone produced at various stages of an animal's sexual development. Most of the chemicals proposed for such sterilization purposes are hormones or hormone analogs. For example U.S. Pat. No. 4,444,759 describes a class of peptides analogous to GnRH (i.e., gonadotropin-releasing hormone, and particularly luteinizing hormone-releasing hormone) capable of inhibiting release of gonadotropins by the pituitary gland and thereby inhibiting release of the steroidal hormones, estradiol, progesterone and testosterone.

Another approach has been to use certain chemicals to produce antibodies in an animal which exhibit cross-reactivity with the gonadotropins produced by the animal's pituitary gland. It is generally thought that with such early antigenic stimulation, formation of antibodies is more continuously stimulated by the release of endogenous hormones and that early immunization with such luteinizing hormone deters the maturation of the gonads and adnexal glands. This, in turn, is thought to inhibit spermatogenesis at the spermatogonial level. For example, U.S. Pat. No. 4,691,006 teaches injection of a compound to elicit formation of antibodies which exhibit cross-reactivity with the gonadotropins produced by the animal's pituitary. With early antigenic stimulation of this kind, the formation of such antibodies is more continuously stimulated by release of endogenous hormones. Early immunization with such luteinizing hormone also deters the maturation of the gonads and adnexal glands.

Similarly, luteinizing hormone has been administered to animals after they have attained the age of puberty in order to atrophy their reproductive organs and to cause a decrease in libido (see generally, Tallau and Laurence, Fertility and Sterility, 22 (2): 113-118 (1971); Pineda, et al. Proc. Soc. Exper. Biol. Med. 125 (3):665-668 (1967), and Quadri, et al. Proc. Soc. Exp. Biol. Med., 123:809-814 (1966)). Such treatments also impair spermatogenesis in noncastrated adult male animals by interruption of the spermatogenic cycle.

Other chemical sterilization agents have been specifically designed for use on female animals. For example, it is well known that certain antigens will produce an antiserum against a requisite estrogen. This is accomplished by first making an antigen and then injecting the antigen into an animal for purposes of antiserum production. The animal is then bled to recover the antiserum. Any female animal of the same species as the host animal may then be injected with the antiserum at the proper time prior to ovulation and the injected antiserum will cause temporary sterilization of that animal.

Other methods of chemical sterilization have been based upon direct chemical attack upon certain cells of the pituitary itself (as opposed to chemical attacks upon the hormone products of such cells) with a view toward permanently destroying such cells. Again, this approach is suggested by the fact that follicle stimulating hormone (FSH) and luteinizing hormone (LH) (sometimes referred to as gonadotropins or gonadotropic hormones) are released by the pituitary gland to regulate functioning of the gonads to produce testosterone in the testes and progesterone and estrogen in the ovaries. They also regulate the production and maturation of gametes.

Several chemical agents have been proposed for such purposes. However, it has been found that most chemical agents which are in fact capable of destroying the gonadotrophs of an animal's anterior pituitary gland also tend to produce extremely toxic side effects which can severely weaken, and sometimes kill, the treated animal. Hence, with respect to the general subject of chemical sterilization, any chemical capable of producing sterilization without, or with minimal, toxic side effects would be of great value in the fields of animal husbandry, veterinary medicine and wildlife control.

Myers, et al. Biochem. J., 227 (1):343 (1985) describes a sterilization procedure employing a GnRH/diphtheria toxin conjugate. Singh, et al., Int. J. Pharm. 76: R5-R8 describes controlled release of LHRH-DT from bioerodible hydrogel microspheres which induces production of antibodies to GnRH which then serve to inactivate endogenous LHRH in the circulation. U.S. Pat. No. 6,326,467 to Nett, et al., describes the use of conjugates of cytotoxic compounds including daunomycin and other toxins to analogs of gonadotropin-releasing hormones for sterilization of animals or to treat certain sex hormone related diseases.

It is therefore an object of the present invention to provide compositions and methods for delivery of targeted drugs, especially cytotoxic compounds targeted to cell by specific hormones, for sterilization and treatment of disorders, especially of reproductive tissues.

### SUMMARY OF THE INVENTION

A method and compositions for targeted drug delivery have been developed. The compositions include a targeting molecule, such as a hormone that specifically binds to a receptor on the surface of the targeted cells; a drug to be delivered, such as a toxin that will kill the targeted cells; and a nanoparticle, which contains on or within the nanoparticle, the drug to be delivered, as well as has attached thereto, the targeting molecule. In a preferred embodiment, the ratio of targeting molecule to toxin or other drug is in the range of 1:10 or more. This higher density of toxin is critical to achieving the desired treatment in some cases.

Examples of targeting molecules include hormones, ligands for specific cell surface receptors, and antibodies. Examples of cytotoxic drugs include toxins, BCNU, adriamycin, cisplatin, and other chemotherapeutic agents, radioactive compounds, radioisotopes, especially yttrium (“Y”), and substances which elicit the host to attack tumor cells, and radioactive colloids. Nanoparticles can consist of drug or drug associated with carrier, such as a controlled or sustained release materials like a poly(lactide-co-glycolide), a liposome or surfactant.

The targeted nanoparticles can be administered in combination with compositions for improving cellular internalization using a receptor mediated mechanism are disclosed.

The compositions are administered by injections in most cases, although materials combined with the gel will typically be applied to cell membranes to achieve high rates of transport of the compound to be delivered across those membranes, relative to when non-viscous fluids are used with the enhancers or the viscous fluids are used alone. Compositions can be applied topically orally, nasally, vaginally, rectally, and ocularly. Enhancer is administered systemically or, more preferably, locally. Compositions can be applied by injection via catheter, intramuscularly, subcutaneously, and intraperitoneally. Compositions can also be administered to the pulmonary or respiratory system, most preferably in an aerosol.

### BRIEF DESCRIPTION OF THE DRAWINGS

FIGS. 1A and 1B are graphs of the association of florescence nanoparticles, with and without LHRH, with cells as determined by flow cytometry. FIG. 1A illustrates intensity of cell florescence (log) obtained when incubating cells with nanoparticles not conjugated to LHRH as described in Example 2. FIG. 1B shows the increased intensity of cellular florescence achieved following exposure of cells to LHRH-conjugated florescence nanoparticles.

### DETAILED DESCRIPTION OF THE INVENTION

A method and compositions have been developed in which a drug is targeted to a specific cell via a receptor on the surface of the targeted cells. The drug is encapsulated within and/or bound to a nanoparticle to which the targeting molecule is also bound. The compositions are then used to deliver medication to a specific group of cells in the body. Hormones are a preferred targeting molecule because they travel throughout the systemic circulation and thus reach all parts of the body; however, they have physiological effects only on cells which are capable of ‘recognizing’ their proximity through the presence of biological ‘receptors’ embedded within the cell membrane. Each hormone has its own specific type of receptor; insulin, for example, will only cause a change in biological function in cells that possess specific insulin receptors.

In some cases, a biological change in cell function is effected as soon as the hormone attaches to its receptor on the membrane; in other cases, physiological effects will only be observed when the hormone attaches to the receptor and, additionally, enters the cell. This latter process is known as ‘receptor-mediated endocytosis’ and is the key to highly selective hormonal targeting of certain cell types. For example, the method takes advantage of the fact that in all mammals, cells in the anterior pituitary gland naturally recognize and absorb a hormone called luteinizing-hormone-releasing-hormone (LHRH). The anterior pituitary cells (known as gonadotrope cells) are subsequently responsible for the release into the bloodstream of hormones that enable sexual function. The method and compositions essentially uses LHRH as a ‘key’ to unlock a pathway into these cells. Attached to the key is a polymer ‘keychain’, and swinging on the end of that keychain is a ‘payload’ in the form of a tiny complex called a nanoparticle. The nanoparticle can be thought of as a tiny spherical shell; encapsulated within that shell is a toxin that is to be delivered to the gonadotrope cells. The nanoparticle by definition will be extremely small in relation to the cell being entered. It enters the cell along with its toxic payload and then degrades after a time that can be predetermined when engineering the nanoparticle in the laboratory. As it degrades, the nanoparticle releases its payload and the toxin destroys the gonadotrope cell. The death of the cell means that it can no longer produce the hormones that enable sexual function. Without the presence of those hormones in the body, the animal is effectively sterile, or, in the case of a tumor of a reproductive tissue such as the testes, prostate, or ovaries, the tumor is killed. Since the pituitary cells do not regrow with time, this is an irreversible procedure that is marketable as a humane and surgery-free alternative to ovariectomies and orchiectomies currently performed by veterinary surgeons. The gonadotrope cells are the only known cells within the mammalian body that express receptors for LHRH. The compound does not cross the blood-brain barrier.

The nanoparticle both protects the chemical toxin from being degraded before it reaches the target cell and ensures there is no ‘leakage’ of the chemical into the bloodstream, thereby protecting other cells from its harmful effects. Additionally, the tiny dimensions of the nanoparticle serve to make certain that it will be carried into the target cell alongside the LHRH without difficulty. The size of the nanoparticle can be engineered to enhance uptake of the toxin into the cell.

Other embodiments are created by substituting another hormone in order to target other cells, and other kinds of drugs. One area is in the targeting of human cancerous cells in malignant tumors. It is known that growing cancer cells show an inordinately high number of transferrin receptors (on the order of fifteen times the number found on healthy cells).

In still another embodiment, delivery is enhanced through the use of hydrogels of a certain viscosity to enable transport of drugs across epithelial membranes. If one adds a hydrogel of viscosity identical to that of the epithelial cell cytoplasm, the resulting rate of transport of LHRH into the cell and across the epithelial barrier will be optimized. This opens up the potential for an application along the lines of an eye gel used to treat glaucoma. A drug such as epinephrin (a recognized glaucoma treatment) could be locked inside a nanoparticle and then the original LHRH key could carry it into the eye and to the active drug site within the nasolacrimal gland. Topical eye medications have traditionally been difficult to design because they are washed out of the eye so quickly; a hydrogel would enhance the rate at which the drug enters the system and thus markedly reduce the number of drug applications required to control the disease. At present, patients are required to use eye drops or creams as often as once every six hours to ensure the intra-ocular pressure is maintained at a safe level.

I. Compositions

A. Agents to be Delivered

Therapeutic, diagnostic or prophylactic agents can be specifically delivered using the technology described herein.

Any of a variety of therapeutic, diagnostic or prophylactic agents can be incorporated within the particles, or used to prepare particles consisting solely of the agent and surfactant. The particles can be used to locally or systemically deliver a variety of incorporated agents to a targeted tissue of an animal. Examples include synthetic inorganic and organic compounds, proteins and peptides, polysaccharides and other sugars, lipids, and DNA and RNA nucleic acid sequences having therapeutic, prophylactic or diagnostic activities. Nucleic acid sequences include genes, antisense molecules which bind to complementary DNA to inhibit transcription, and ribozymes. The agents to be incorporated can have a variety of biological activities, such as vasoactive agents, neuroactive agents, hormones, anticoagulants, immunomodulating agents, cytotoxic agents, prophylactic agents, antibiotics, antivirals, antisense, antigens, and antibodies. In some instances, the proteins may be antibodies or antigens which otherwise would have to be administered by injection to elicit an appropriate response. Compounds with a wide range of molecular weight can be encapsulated, for example, between 100 and 500,000 grams or more per mole. As used herein, proteins are defined as consisting of 100 amino acid residues or more; peptides are less than 100 amino acid residues. Unless otherwise stated, the term protein refers to both proteins and peptides. Examples include insulin and other hormones. Polysaccharides, such as heparin, can also be administered.

Examples of cytotoxic drugs include toxins, BCNU, adriamycin, cisplatin, and other chemotherapeutic agents, radioactive compounds, radioisotopes, especially yttrium (“Y”), and substances which elicit the host to attack tumor cells (Halpern, et al., J. Nucl. Med. 29:1688-1696 (1988); Quadri, et al., Nucl. Med. Biol. 20:559-570 (1993); Wang, et al., Radiat. Res. 141:292-302 (1995)), oligonucleotides (Mujoo, et al., Oncogene 12:1617-1623 (1996)), cytokines (Markman, Semin. Oncol. 18:248-254 (1991); Dedrick, et al., Cancer. Treat Rep. 62:1-11 (1978)), and radioactive colloids (Rowlinson, et al., Cancer Res. 47:6528-6531 (1987)). Preferred toxins include: diphtheria toxin, ricin toxin, abrin toxin, pseudomonas exotoxin, shiga toxin, .alpha.-amanitin, pokeweed antiviral protein (PAP), ribosome inhibiting proteins (RIP), especially the ribosome inhibiting proteins of barley, wheat, flax, corn, rye, gelonin, abrin, modeccin and certain cytotoxic chemicals such as, for example, melphalan, methotrexate, nitrogen mustard, doxorubicin and daunomycin. These may be administered as intact molecules or as subunits, as appropriate. TABLE I below gives some representative “whole” and “modified” toxins. Some of these toxin types (e.g., bacterial and plant toxins) also can be further characterized by their possession of so-called “A-chain” and “B-chain” groups in their molecular structures. It also should be noted that the toxic domain is often referred to as the “A-chain” portion of the toxin molecule while the toxic domain, translocation domain and cell-binding domain are often collectively referred to as the “whole” toxin or the A-chain plus the B-chain molecules. For example, such further classifications could be made according to the attributes, categories and molecular sizes noted in TABLE I below (wherein the letters A and B represent the presence of A-chains or B-chains and the letter K designates the symbol (“kilodalton” used to designate molecular sizes of such molecules):

TABLE I
Toxins

Single Chain Toxins
Pokeweed antiviral protein
Gelonin ribosome-inhibiting protein (RIP)
Wheat RIP
Barley RIP
Corn RIP
Rye RIP
Flax RIP
Bacterial Toxins
Diphtheria toxin (whole) having a toxic domain, a translocation
domain and a cell-binding domain = 62K
Diphtheria toxin (modified) having a toxic domain and a
translocation domain = 45K
Pseudomonas exotoxin (whole) having a toxic domain, a
translocation domain and a cell-binding domain = 66K
Pseudomonas exotoxin (modified) having a toxic domain and a
translocation domain = 40K
Shiga toxin (whole) having a toxic domain, a translocation domain
and a cell binding domain = 68K
Shiga toxin (modified) having a toxic domain = 30K
Plant Toxins
Ricin A + B (whole) = 62K
Ricin A = 30K
Abrin A + B = 62K
Abrin A = 30K
Modeccin A + B = 56K
Modeccin A = 26K
Small Chemical Toxins
Melphalan
Methotrexate
Nitrogen Mustard
Daunomycin
Doxorubicin

Hormones include peptide-releasing hormones such as insulin, luteinizing hormone releasing hormone (“LHRH”), gonadotropin releasing hormone (“GnRH”), deslorelin and leuprolide acetate, oxytocin, vasoactive intestinal peptide (VIP), glucagon, parathyroid hormone (PTH), thyroid stimulating hormone, follicle stimulating hormone, growth factors such as nerve growth factor (NGF), epidermal growth factor (EGF), vascular endothelial growth factor (VEGI), insulin-like growth factors (IGF-I and IGF-II), fibroblast growth factors (FGFs), platelet-derived endothelial cell growth factor (PD-ECGF), transforming growth factor beta (TGF-β), and keratinocyte growth factor (KGF). Other materials which can be delivered include cytokines such as tumor necrosis factors (TFN-α and TNF-β), colony stimulating factors (CSFs), interleukin-2, gamma interferon, consensus interferon, alpha interferons, beta interferon; attachment peptides such as RGD; bioactive peptides such as renin inhibitory peptides, vasopressin, detirelix, somatostatin, and vasoactive intestinal peptide; coagulation inhibitors such as aprotinin, heparin, and hirudin; enzymes such as superoxide dismutase, neutral endopeptidase, catalase, albumin, calcitonin, alpha-1-antitrypsin (A1A), deoxyribonuclease (DNAase), lectins such as concanavalin A, and analogues thereof.

Diagnostic agents can also be delivered. These can be administered alone or coupled to one or more bioactive compounds as described above. The agents can be radiolabelled, fluorescently labeled, enzymatically labeled and/or include magnetic compounds and other materials that can be detected using x-rays, ultrasound, magnetic resonance imaging (“MRI”), computed tomography (“CT”), or fluoroscopy.

Genes for the treatment of diseases such as cystic fibrosis can be administered, as can beta agonists for asthma.

Those therapeutic agents which are charged, such as most of the proteins, including insulin, can be administered as a complex between the charged therapeutic agent and a molecule of opposite charge. Preferably, the molecule of opposite charge is a charged lipid or an oppositely charged protein.

Any of a variety of diagnostic agents can be incorporated within the particles, which can locally or systemically deliver the incorporated agents following administration to a patient. Any biocompatible or pharmacologically acceptable gas can be incorporated into the particles or trapped in the pores of the particles using technology known to those skilled in the art. The term gas refers to any compound which is a gas or capable of forming a gas at the temperature at which imaging is being performed. In one embodiment, retention of gas in the particles is improved by forming a gas-impermeable barrier around the particles. Such barriers are well known to those of skill in the art.

Other imaging agents which may be utilized include commercially available agents used in positron emission tomography (PET), computer assisted tomography (CAT), single photon emission computerized tomography, x-ray, fluoroscopy, and magnetic resonance imaging (MRI).

Examples of suitable materials for use as contrast agents in MRI include the gadolinium chelates currently available, such as diethylene triamine pentacetic acid (DTPA) and gadopentotate dimeglumine, as well as iron, magnesium, manganese, copper and chromium.

Examples of materials useful for CAT and x-rays include iodine based materials for intravenous administration, such as ionic monomers typified by diatrizoate and iothalamate, non-ionic monomers such as iopamidol, isohexol, and ioversol, non-ionic dimers, such as iotrol and iodixanol, and ionic dimers, for example, ioxagalte.

Excipients

In addition to a therapeutic or diagnostic agent (or possibly other desired molecules for delivery), the particles can include, and preferably, do include, one or more of the following excipients; a sugar, such as lactose, a protein, such as albumin, and/or a surfactant.

B. Carriers

The drug and targeting molecule are linked to a common carrier, preferably a nanoparticle, which must be able to bind to a cell surface receptor. As used herein, a “nanoparticle” includes liposomes, micelles, drug particles and polymeric particles, having a dimension of less than one micron, more preferably in the range of less than or equal to 200 nm, although nanoparticles may be in the range of as large as approximately 500 nm, and as small as a few nm. Liposomes consist basically of a phospholipid bilayer forming a shell around an aqueous core. Advantages include the lipophilicity of the outer layers which “mimic” the outer membrane layers of cells and that they are taken up relatively easily by a variety of cells. Polymeric vehicles typically consist of nanospheres and nanocapsules formed of biocompatible polymers, which are either biodegradable (for example, polylactic acid) or non-biodegradable (for example, ethylenevinyl acetate). Some of the advantages of the polymeric devices are ease of manufacture and high loading capacity, range of size from nanometer to micron diameter, as well as controlled release and degradation profile. Both liposomes and small polymeric vehicles are referred to herein as “nanoparticles”, unless specifically stated otherwise.

The particles can be prepared entirely from a therapeutic or diagnostic agent, or from a combination of the agent and a surfactant, excipient or polymeric material. The particles preferably are biodegradable and biocompatible, and optionally are capable of biodegrading at a controlled rate for delivery of a therapeutic or diagnostic agent. The particles can be made of a variety of materials. Both inorganic and organic materials can be used. Polymeric and non-polymeric materials, such as fatty acids, may be used. Other suitable materials include, but are not limited to, gelatin, polyethylene glycol, trehalose, and dextran. Particles with degradation and release times ranging from seconds to months can be designed and fabricated, based on factors such as the particle material.

Polymeric Particles

Polymeric particles may be formed from any biocompatible, and preferably biodegradable polymer, copolymer, or blend. The polymers may be tailored to optimize different characteristics of the particle including: i) interactions between the agent to be delivered and the polymer to provide stabilization of the agent and retention of activity upon delivery; ii) rate of polymer degradation and, thereby, rate of drug release profiles; iii) surface characteristics and targeting capabilities via chemical modification; and iv) particle porosity.

Surface eroding polymers such as polyanhydrides may be used to form the particles. For example, polyanhydrides such as poly[(p-carboxyphenoxy)-hexane anhydride] (PCPH) may be used. Biodegradable polyanhydrides are described in U.S. Pat. No. 4,857,311.

In another embodiment, bulk eroding polymers such as those based on polyesters including poly(hydroxy acids) can be used. For example, polyglycolic acid (PGA), polylactic acid (PLA), or copolymers thereof may be used to form the particles. The polyester may also have a charged or functionalizable group, such as an amino acid. In a preferred embodiment, particles with controlled release properties can be formed of poly(D,L-lactic acid) and/or poly(D,L-lactic-co-glycolic acid) (“PLGA”) which incorporate a surfactant such as DPPC.

Other polymers include polyamides, polycarbonates, polyalkylenes such as polyethylene, polypropylene, poly(ethylene glycol), poly(ethylene oxide), poly(ethylene terephthalate), poly vinyl compounds such as polyvinyl alcohols, polyvinyl ethers, and polyvinyl esters, polymers of acrylic and methacrylic acids, celluloses and other polysaccharides, and peptides or proteins, or copolymers or blends thereof. Polymers may be selected with or modified to have the appropriate stability and degradation rates in vivo for different controlled drug delivery applications.

In one embodiment, particles are formed from functionalized polyester graft copolymers, as described in Hrkach et al., Macromolecules, 28:4736-4739 (1995); and Hrkach et al., “Poly(L-Lactic acid-co-amino acid) Graft Copolymers: A Class of Functional Biodegradable Biomaterials” in Hydrogels and Biodegradable Polymers for Bioapplications, ACS Symposium Series No. 627, Raphael M. Ottenbrite et al., Eds., American Chemical Society, Chapter 8, pp. 93-101, 1996.

Materials other than biodegradable polymers may be used to form the particles. Suitable materials include various non-biodegradable polymers and various excipients. The particles also may be formed of a therapeutic or diagnostic agent and surfactant alone. In one embodiment, the particles may be formed of the surfactant and include a therapeutic or diagnostic agent.

Polymeric particles may be prepared using single and double emulsion solvent evaporation, spray drying, solvent extraction, solvent evaporation, phase separation, simple and complex coacervation, interfacial polymerization, and other methods well known to those of ordinary skill in the art. Particles may be made using methods for making microspheres or microcapsules known in the art, provided that the conditions are optimized for forming particles with the desired diameter.

Methods developed for making microspheres for delivery of encapsulated agents are described in the literature, for example, as described in Doubrow, M., Ed., “Microcapsules and Nanoparticles in Medicine and Pharmacy,” CRC Press, Boca Raton, 1992. Methods also are described in Mathiowitz and Langer, J. Controlled Release 5,13-22 (1987); Mathiowitz et al., Reactive Polymers 6, 275-283 (1987); and Mathiowitz et al., J. Appl. Polymer Sci. 35, 755-774 (1988). The selection of the method depends on the polymer selection, the size, external morphology, and crystallinity that is desired, as described, for example, by Mathiowitz et al., Scanning Microscopy 4: 329-340 (1990); Mathiowitz et al., J. Appl. Polymer Sci. 45, 125-134 (1992); and Benita et al., J. Pharm. Sci. 73, 1721-1724 (1984).

In solvent evaporation, described for example, in Mathiowitz et al., (1990), Benita; and U.S. Pat. No. 4,272,398 to Jaffe, the polymer is dissolved in a volatile organic solvent, such as methylene chloride. Several different polymer concentrations can be used, for example, between 0.05 and 1.0 g/ml. The therapeutic or diagnostic agent, either in soluble form or dispersed as fine particles, is added to the polymer solution, and the mixture is suspended in an aqueous phase that contains a surface active agent such as poly(vinyl alcohol). The aqueous phase may be, for example, a concentration of 1% poly(vinyl alcohol) w/v in distilled water. The resulting emulsion is stirred until most of the organic solvent evaporates, leaving solid microspheres, which may be washed with water and dried overnight in a lyophilizer. Microspheres with different sizes (between 1 and 1000 microns) and morphologies can be obtained by this method.

Solvent removal was primarily designed for use with less stable polymers, such as the polyanhydrides. In this method, the agent is dispersed or dissolved in a solution of a selected polymer in a volatile organic solvent like methylene chloride. The mixture is then suspended in oil, such as silicon oil, by stirring, to form an emulsion. Within 24 hours, the solvent diffuses into the oil phase and the emulsion droplets harden into solid polymer microspheres. Unlike the hot-melt microencapsulation method described for example in Mathiowitz et al., Reactive Polymers, 6:275 (1987), this method can be used to make microspheres from polymers with high melting points and a wide range of molecular weights. Microspheres having a diameter for example between one and 300 microns can be obtained with this procedure.

With some polymeric systems, polymeric particles prepared using a single or double emulsion technique vary in size depending on the size of the droplets. If droplets in water-in-oil emulsions are not of a suitably small size to form particles with the desired size range, smaller droplets can be prepard, for example, by sonication or homogenation of the emulsion, or by the addition of surfactants.

If the particles prepared by any of the above methods have a size range outside of the desired range, particles can be sized, for example, using a sieve, and further separated according to density using techniques known to those of skill in the art.

The polymeric particles can be prepared by spray drying. Methods of spray drying, such as that disclosed in PCT WO 96/09814 by Sutton and Johnson, disclose the preparation of smooth, spherical microparticles of a water-soluble material with at least 90% of the particles possessing a mean size between 1 and 10 μm.

Liposomes

Liposomes can be produced by standard methods such as those reported by Kim, et al., Biochim. Biophys. Acta 728, 339-348 (1983); Liu, D., et al., Biochim. Biophys. Acta 1104, 95-101 (1992); and Lee, et al., Biochim. Biophys. Acta., 1103, 185-197 (1992)). Many liposome formulations using many different lipid components have been used in various in vitro cell culture and animal experiments. Parameters have been identified that determine liposomal properties and are reported in the literature, for example, by Lee, K. D., et al. Biochim. Biophys. Acta., 1103, 185-197 (1992); Liu, D., Mori, A. and Huang, L., Biochim. Biophys. Acta, 1104, 95-101 (1992); Wang, C. Y. and Huang, L., Biochem., 28, 9508-9514 (1989)).

Briefly, the lipids of choice, dissolved in an organic solvent, are mixed and dried onto the bottom of a glass tube under vacuum. The lipid film is rehydrated using an aqueous buffered solution containing the material to be encapsulated by gentle swirling. The hydrated lipid vesicles or liposomes are washed by centrifugation and can be filtered and stored at 4° C. This method is described in more detail in Thierry, A. R. and Dritschilo, A “Intracellular availability of unmodified, phosphorothioated and liposomally encapsulated oligodeoxynucleotides for antisense activity” Nuc. Ac. Res. 20:5691-5698 (1992).

A nanoparticle system carrying a toxin payload can be made using the procedure as described in: Pautot, Frisken, Weitz “Production of unilamellar vesicles using an inverted emulsion” (submitted for publication). Using Pautot et al's technique, streptavidin-coated lipids (DPPC, DSPC, and similar lipids) can be used to manufacture liposomes. The drug encapsulation technique described by Needham D, Newhirst M W. Advanced Drug Delivery Reviews, 53 (3): 285-305 Dec. 31 2001), can be used to load these vesicles with doxorubicin, a model small-molecule toxin currently used in cancer therapy.

The liposomes can be prepared by exposing chloroformic solution of various lipid mixtures to high vacuum and subsequently hydrating the resulting lipid films (DSPC/CHOL) with pH 4 buffers, and extruding them through polycarbonated filters, after a freezing and thawing procedure. It is possible to use DPPC supplemented with DSPC or cholesterol to increase encapsulation efficiency or increase stability, etc. A transmembrane pH gradient is created by adjusting the pH of the extravesicular medium to 7.5 by addition of an alkalinization agent. The anticancer drug doxorubicin is subsequently entrapped by addition of the drug solution in small aliquots to the vesicle solution, at an elevated temperature, to allow drug accumulation inside the liposomes.

Complex Forming Materials

If the agent to be delivered is negatively charged (such as insulin), protamine or other positively charged molecules can be added to provide a lipophilic complex which results in the sustained release of the negatively charged agent. Negatively charged molecules can be used to render insoluble positively charged agents.

Materials Enhancing Sustained Release

If the molecules are hydrophilic and tend to solubilize readily in an aqueous environment, another method for achieving sustained release is to use cholesterol or very high surfactant concentration.

Surfactants

Surfactants which can be incorporated into particles include phosphoglycerides. Exemplary phosphoglycerides include phosphatidylcholines, such as the naturally occurring surfactant, L-α-phosphatidylcholine dipalmitoyl (“DPPC”). The surfactants advantageously improve surface properties by, for example, reducing particle-particle interactions, and can render the surface of the particles less adhesive. The use of surfactants endogenous to the lung may avoid the need for the use of non-physiologic surfactants.

As used herein, the term “surfactant” refers to any agent which preferentially absorbs to an interface between two immiscible phases, such as the interface between water and an organic polymer solution, a water/air interface or organic solvent/air interface. Surfactants generally possess a hydrophilic moiety and a lipophilic moiety, such that, upon absorbing to microparticles, they tend to present moieties to the external environment that do not attract similarly-coated particles, thus reducing particle agglomeration. Surfactants may also promote absorption of a therapeutic or diagnostic agent and increase bioavailability of the agent.

As used herein, a particle “incorporating a surfactant” refers to a particle with a surfactant on at least the surface of the particle. The surfactant may be incorporated throughout the particle and on the surface during particle formation, or may be coated on the particle after particle formation. The surfactant can be coated on the particle surface by adsorption, ionic or covalent attachment, or physically “entrapped” by the surrounding matrix. The surfactant can be, for example, incorporated into controlled release particles, such as polymeric microspheres.

Providing a surfactant on the surfaces of the particles can reduce the tendency of the particles to agglomerate due to interactions such as electrostatic interactions, Van der Waals forces, and capillary action. The presence of the surfactant on the particle surface can provide increased surface rugosity (roughness), thereby improving aerosolization by reducing the surface area available for intimate particle-particle interaction. The use of a surfactant which is a natural material of the lung can potentially reduce opsonization (and thereby reducing phagocytosis by alveolar macrophages), thus providing a longer-lived controlled release particle in the lung.

Surfactants known in the art can be used including any naturally occurring surfactant. Other exemplary surfactants include diphosphatidyl glycerol (DPPG); hexadecanol; fatty alcohols such as polyethylene glycol (PEG); polyoxyethylene-9-lauryl ether; a surface active fatty acid, such as palmitic acid or oleic acid; sorbitan trioleate (Span 85); glycocholate; surfactin; a poloxomer; a sorbitan fatty acid ester such as sorbitan trioleate; tyloxapol and a phospholipid.

C. Targeting of Particles

The nanoparticles are targeted so that they are delivered to a particular cell type. It is well known in the art how to modify carriers such that they are bound, ionically or covalently, to a ligand (i.e., LHRH) that binds to a cell surface receptor. Examples of materials used to covalently bind targeting molecules to the materials forming the carrier including 2-iminothiolane, N-succinimidyl-3-(2-pyridyldithio)proprionate (SPDP), 4-succinimidyloxycarbonyl-.alpha.-(2-pyridyldithio)-toluene (SMPT), m-maleimidobenzoyl-N-hydroxysuccinimide-ester (MBS), N-succinimidyl(4-iodoacetyl)aminobenzoate (SIAB), succinimidyl 4-(p-maleimidophenyl)butyrate (SMPB), 1-ethyl-3-(3-dimethylaminopropyl)carbodiimide (EDC), bis-diazobenzidine and glutaraldehyde. Other means of coupling targeting molecules can be used with materials such as liposomes. For example, U.S. Pat. No. 5,258,499 to Konigsberg et al. describes the incorporation of receptor specific ligands into liposomes, which are then used to target receptors on the cell surface. The use of carriers can be particularly important for intracellularly delivering nucleic acid molecules. In one embodiment, nucleic acid molecules are encapsulated in a liposome, preferably a cationic liposome, that has a receptor-binding ligand, such as LHRH, on its surface. The liposome is then dispersed in a viscous fluid. When the composition is administered, the liposomes are endocytosed by the cell, and the nucleic acid molecules are released from the liposome inside the cell.

Examples of targeting molecules include hormones, ligands for specific cell surface receptors, and antibodies. A key criteria is that the targeting molecule specifically binds to a receptor on the surface of the cells where the drug is to be delivered. Moreover, the targeting molecule should preferably assist in uptake of the drug to be delivered into the cell, as in the case of hormone binding to receptors which result in endocytosis of the nanoparticle containing the drug into the cell.

Targeting molecules can be attached to the particles via reactive functional groups on the particles. For example, targeting molecules can be attached to the amino acid groups of functionalized polyester graft copolymer particles, such as poly(lactic acid-co-lysine) (PLAL-Lys) particles. Targeting molecules permit binding interaction of the particle with specific receptor sites, such as those within the lungs. The particles can be targeted by attachment of ligands which specifically or non-specifically bind to particular targets. Exemplary targeting molecules include antibodies and fragments thereof including the variable regions, lectins, and hormones or other organic molecules capable of specific binding, for example, to receptors on the surfaces of the target cells.

The binding of ligands or assembly proteins to surface receptors of eucaryotic cell membranes has been extensively studied in an effort to develop better ways to promote or enhance cellular uptake. For example, binding of ligands or proteins has been reported to initiate or accompany a cascade of nonequilibrium phenomena culminating in the cellular invagination of membrane complexes within clathrin-coated vesicles (Goldstein, et al., Ann. Rev. Cell Biol. 1:1-39 (1985); Rodman, et al., Curr. Op. Cell Biol. 2:664-72 (1990); Trowbridge, Curr. Op. Cell Biol. 3:634-41 (1991); Smythe, et al., J. Cell Biol. 108:843-53 (1989); Smythe, et al., J. Cell Biol. 119:1163-71 (1992); and Schmid, Curr. Op. Cell Biol. 5:621-27 (1993)). This process has been referred to as receptor-mediated endocytosis (“RME”). Beyond playing a central role in cellular lipid trafficking (Pagano, Curr. Op. Cell Biol. 2:652-63 (1990)), RME is the primary means by which macromolecules enter eucaryotic cells. An effective strategy for enhancing the uptake of cytotoxic and therapeutic drugs involves exploiting the rapidity and specificity of transmembrane transport via receptor-mediated endocytosis (Goldstein, et al., Ann. Rev. Cell Biol. 1:1-39 (1985)) by targeting receptors on the plasma membranes of endothelial (Barzu, et al., Biochem. J. 15; 238 (3):847-854 (1986); Magnusson & Berg, Biochem. J. 257:65-56 (1989)), phagocytic (Wright & Detmers, “Receptor-mediated phagocytosis” in The Lung: Scientific Foundations (Crystal, et al., eds.), pp. 539-49 (Ravens Press, Ltd., New York, N.Y. (1991)); and tumor cells, as well as cells of other tissues.

D. Gels for Enhancement of Uptake into Cells.

It has been demonstrated that, by embedding individual cell populations in hydrogel media of macroscopic viscosity similar to that characteristic of cell cytoskeleta, the rate of receptor-mediated endocytosis can be significantly enhanced (Edwards, et al., Proc. Natl. Acad. Sci. U.S.A. 93:1786-91 (1996); PCT US97/03276 by Massachusetts Institute of Technology and Pennsylvania State University Foundation). This enhancement effect appears to reflect a fluid-mechanical origin of receptor-mediated endocytosis, involving the rapid expansion of plasma membrane in the vicinity of a receptor cluster leading to an invaginating membrane motion that is sensitive to the viscous properties of the extracellular environment (Edwards, et al., Proc. Natl. Acad. Sci. U.S.A. 93:1786-91 (1996); Edwards, et al., Biophys. J. 71:1208-14 (1996)).

When the differences between the apparent viscosities of the cytosolic fluid and the extracellular fluid are extremely large, membrane deformation is strongly resisted and the initial thrust of the membrane is damped. However, as the differences between the apparent viscosities of the cytosolic fluid and the extracellular fluid become extremely small, membrane deformation becomes progressively rapid. Accordingly, the rate of endocytosis can be increased by adjusting the viscosity of the extracellular fluid so that it is approximately the same as that of the cytosolic fluid, as described by PCT/US97/03276. If the viscosity of the extracellular fluid is appreciably higher or lower than that of the cytosolic fluid, the rate of endocytosis decreases.

Clustering of membrane complexes is favorable for rapid internalization. The rate of internalization can be increased in proportion to the magnitude of binding energy. This is due, in part, to the specificity of receptors to particular ligands and/or adaptor proteins. Clustering of complexes occurs in the vicinity of pits to which clathrin triskelions absorb from the cytosolic side of the cell membrane and subsequently polymerize to form a clathrin coat. Some clustering has also been observed in the vicinity of caveolae, or non-clathrin-coated pits. The membrane-tension depression occurring within the vicinity of an evolving pit, originating in the process of membrane complexation, is directly proportional to the number of membrane complexes formed within that pit. In general, clustered complexes have been found to internalize substances more rapidly than nonclustered complexes.

The magnitudes of apparent viscosity difference and receptor clustering have each been found to alter the rate of RME. Membrane tension can also be manipulated to influence the rate of RME. Increasing the membrane tension ‘hardens’ the cell membrane, making cell membrane depression increasingly prohibitive. This phenomenon has been commented upon by Sheetz, M. P. and Dai, J. (1995), presented at the 60th Annual Cold Spring Harbor Symposium on Protein Kinases, Cold Spring Harbor, N.Y., on the basis of studies that show an increased rate of endocytosis for neuronal growth cones coinciding with membrane tension lowering.

Accordingly, the rate of internalization can be increased by a) adjusting the viscosity of the extracellular fluid to approximate that of the cytosolic fluid; b) forming complexes of the material to be internalized; and c) reducing membrane tension. Compositions and methods for increasing the rate of endocytosis are described in detail below.

Suitable viscous fluids for use in intracellularly administering compounds include biocompatible hydrogels, lipogels, and highly viscous sols. A hydrogel is defined as a substance formed when an organic polymer (natural or synthetic) is cross-linked via covalent, ionic, or hydrogen bonds to create a three-dimensional open-lattice structure which entraps water molecules to form a gel. Examples of materials which can be used to form a hydrogel include polysaccharides, proteins and synthetic polymers. Examples of polysaccharides include celluloses such as methyl cellulose, dextrans, and alginate. Examples of proteins include gelatin and hyaluronic acid. Examples of synthetic polymers include both biodegradeable and non-degradeable polymers (although biodegradeable polymers are preferred), such as polyvinyl alcohol, polyacrylamide, polyphosphazines, polyacrylates, polyethylene oxide, and polyalkylene oxide block copolymers (“POLOXAMERS™”) such as PLURONICS™ or TETRONICS™ (polyethylene oxide-polypropylene glycol block copolymers).

In general, these polymers are at least partially soluble in aqueous solutions, such as water, buffered salt solutions, or aqueous alcohol solutions. Several of these have charged side groups, or a monovalent ionic salt thereof. Examples of polymers with acidic side groups that can be reacted with cations are polyphosphazenes, polyacrylic acids, poly(meth)acrylic acids, polyvinyl acetate, and sulfonated polymers, such as sulfonated polystyrene. Copolymers having acidic side groups formed by reaction of acrylic or methacrylic acid and vinyl ether monomers or polymers can also be used. Examples of acidic groups are carboxylic acid groups, sulfonic acid groups, halogenated (preferably fluorinated) alcohol groups, phenolic OH groups, and acidic OH groups.

Examples of polymers with basic side groups that can be reacted with anions are polyvinyl amines, polyvinyl pyridine, polyvinyl imidazole, polyvinylpyrrolidone and some imino substituted polyphosphazenes. The ammonium or quaternary salt of the polymers can also be formed from the backbone nitrogens or pendant imino groups. Examples of basic side groups are amino and imino groups.

Alginate can be ionically cross-linked with divalent cations, in water, at room temperature, to form a hydrogel matrix. An aqueous solution containing the compound to be delivered can be suspended in a solution of a water soluble polymer, and the suspension can be formed into droplets which are configured into discrete microcapsules by contact with multivalent cations. Optionally, the surface of the microcapsules can be crosslinked with polyamino acids to form a semipermeable membrane around the encapsulated materials.

The polyphosphazenes suitable for cross-linking have a majority of side chain groups which are acidic and capable of forming salt bridges with di- or trivalent cations. Examples of preferred acidic side groups are carboxylic acid groups and sulfonic acid groups. Hydrolytically stable polyphosphazenes are formed of monomers having carboxylic acid side groups that are crosslinked by divalent or trivalent cations such as Ca²⁺ or Al³⁺. Polymers can be synthesized that degrade by hydrolysis by incorporating monomers having imidazole, amino acid ester, or glycerol side groups. For example, a polyanionic poly[bis(carboxylatophenoxy)]phosphazene (PCPP) can be synthesized, which is cross-linked with dissolved multivalent cations in aqueous media at room temperature or below to form hydrogel matrices.

Methods for the synthesis of the polymers described above are known to those skilled in the art. See, for example Concise Encyclopedia of Polymer Science and Polymeric Amines and Ammonium Salts, (Goethals, ed.) (Pergamen Press, Elmsford, N.Y. 1980). Many of these polymers are commercially available.

Preferred hydrogels include aqueous-filled polymer networks composed of celluloses such as methyl cellulose, dextrans, agarose, polyvinyl alcohol, hyaluronic acid, polyacrylamide, polyethylene oxide and polyoxyalkylene polymers (“poloxamers”), especially polyethylene oxide-polypropylene glycol block copolymers, as described in U.S. Pat. No. 4,810,503. Several poloxamers are commercially available from BASF and from Wyandotte Chemical Corporation as “Pluronics”. They are available in average molecular weights of from about 1100 to about 15,500.

As used herein, lipogels are gels with nonaqueous fluid interstices. Examples of lipogels include natural and synthetic lecithins in organic solvents to which a small amount of water is added. The organic solvents include linear and cyclic hydrocarbons, esters of fatty acids and certain amines (Scartazzini et al. Phys. Chem. 92:829-33 (1988)). As defined herein, a sol is a colloidal solution consisting of a liquid dispersion medium and a colloidal substance which is distributed throughout the dispersion medium. A highly viscous sol is a sol with a viscosity between approximately 0.1 and 2000 Poise. Other useful viscous fluids include gelatin and concentrated sugar (such as sorbitol) solutions with a viscosity between approximately 0.1 and 2000 Poise.

The apparent viscosity of the extracellular fluid (the composition) must be approximately equal to the viscosity of the cytosolic fluid in the cell to which the compounds are to be administered. One of skill in the art can readily determine or reasonably estimate of the viscosity of the cytosolic fluid using a viscometer and measuring the applied stress divided by measured strain rate at the applied stress that corresponds to the stress the cell membrane imparts upon the cytosolic and extracellular fluids during endocytosis. Methods for measuring the cytosolic viscosity include micropipette methods (Evans & Young, Biophys. J., 56:151-160 (1989)) and methods involving the motion of membrane-linked colloids (Wang et al., Science, 260:1124-26 (1993). Typical cytosol viscosities, measured by these techniques, range from approximately 50-200 Poise. Once this value is measured, the viscosity of the composition can be adjusted to be roughly equal to that viscosity, particularly when measured via routine methods at the applied stress that corresponds to the stress the cell membrane imparts upon the cytosolic and extracellular fluids during endocytosis.

The viscosity can be controlled via any suitable method known to those of skill in the art. The method for obtaining a viscous composition with the desired apparent viscosity is not particularly limited since it is the value of the apparent viscosity relative to the target cells which is critical. The apparent viscosity can be controlled by adjusting the solvent (i.e., water) content, types of materials, ionic strength, pH, temperature, polymer or polysaccharide chemistry performed on the materials, and/or external electric, ultrasound, or magnetic fields, among other parameters.

The apparent viscosity of the compositions is controlled such that it lies in the range of between 0.1 and 2000 Poise, preferably between 7 and 1000 Poise, and most preferably between 2 and 200 Poise. The apparent viscosity can be measured by a standard rheometer using an applied stress range of between 1 and 1000 Pascals, preferably between 1 and 500 Pascals, and most preferably between 1 and 100 Pascals. Further, the viscosity of the compositions is controlled so that the quotient of (apparent viscosity of the cytosol of the target cells—apparent viscosity of the composition) and the apparent viscosity of the cytosol of the target cells is between approximately −0.1 and 0.3, preferably between approximately 0 and 0.3, more preferably between approximately 0 and 0.1, and most preferably between approximately 0 and 0.05.

The composition can be administered as an only slightly viscous formulation that becomes more viscous in response to a condition in the body, such as body temperature or a physiological stimulus, like calcium ions or pH, or in response to an externally applied condition, such as ultrasound or electric or magnetic fields. An example is a temperature sensitive poloxamer which increases in viscosity at body temperature.

The following are examples of suitable concentration ranges: Methocel solutions in the range of between 1.0 and 2.0% (w/w), polyvinyl alcohol solutions between 5 and 15%, pluronic acid solutions between 15 and 20% and trehalose solutions between 1 and 5%.

E. Enhancers

Compounds that can be attached, covalently or noncovalently, to the carrier or nanoparticle that either stimulates receptor-mediated endocytosis (RME) or pinocytosis by binding to receptors on the plasma membrane, binds specifically to receptors that undergo RME or pinocytosis independently of this binding, or at least can be associated chemically or physically with other molecules or “carriers” that themselves undergo RME or pinocytosis, are referred to as enhancers for intracellular delivery. Examples include steroids such as estradiol and progesterone, and some glucocorticoids. Glucocorticocoids such as dexamethasone, cortisone, hydrocortisone, prednisone, and others are routinely administered orally or by injection. Other glucocorticoids include beclomethasone, dipropianate, betamethasone, flunisolide, methyl prednisone, para methasone, prednisolone, triamcinolome, alclometasone, amcinonide, clobetasol, fludrocortisone, diflurosone diacetate, fluocinolone acetonide, fluoromethalone, flurandrenolide, halcinonide, medrysone, and mometasone, and pharmaceutically acceptable salts and mixtures thereof. Other compounds also bind specifically to receptors on cell surfaces. Many hormone specific receptors are known. These can all be used to enhance uptake. Selection of molecules binding to receptors which are predominantly found on a particular cell type or which are specific to a particular cell type can be used to impart selectivity of uptake.

The enhancer is preferably administered at a time and in an amount effective to maximize expression of receptors, and consequently receptor mediated internalization of the compound. The enhancer can itself be the compound to be delivered. The enhancer can be administered as part of the formulation containing the compound to be delivered or prior to or as part of a different formulation. The enhancer may be administered systemically, followed by administration of the compound to be delivered directly to the site where uptake is to occur.

F. Compositions for Lowering or Raising Membrane Tension

The efficiency of the method can be increased by lowering the membrane tension. Suitable methods for lowering membrane tension include including a biocompatible surface active agent in the hydrogel, performing exothermic reactions on the cell surface (i.e., complex formation), and applying an external field to the cell surface. Suitable biocompatible surface active agents include surfactin, trehalose, fatty acids such as palmitin and oleic acid, polyethylene glycol, hexadecanol, and phospholipids such as phosphatidylcholines and phosphatidylglycerols. Suitable complex-forming chemical reactions include the reaction of receptor-binding ligands with cell surface receptors for these ligands, exothermic reactions such as occur between sodium salicylate and salicylic acid, and neutralization reactions as between hydrochloric acid and ammonia (Edwards et al. 1996 Biophys. J. 71, 1208-1214). External fields that can be applied to a cell surface to reduce membrane tension include ultrasound, electric fields, and focused light beams, such as laser beams.

The rate of cellular internalization can also be increased by causing the clustering of receptors on the cell membrane. This can be accomplished, for example, by creating zones on the membrane where the membrane tension is relatively high, causing the membrane fluid to flow toward the zone of high membrane tension. This flow can carry receptors localized in the membrane toward each other, causing them to cluster.

II. Methods of Administration

In the simplest embodiment, the nanoparticle is administered by injection, into the blood stream, peritoneally, subcutaneously, or administered by inhalation, intranasal, intravaginal or topically. The compositions can be applied topically to the vagina, rectum, nose, eye, ear, mouth and the respiratory or pulmonary system. Preferably, the compositions are applied directly to the cells to which the compound is to be delivered, usually in a topical formulation. Examples of methods of administration include oral administration, as in a liquid formulation or within solid foods, topical administration to the skin or the surface of the eye, intravaginal administration, rectal administration, intranasal administration, and administration via inhalation. When the composition is administered orally or by inhalation, it is preferred that it is administered as a dry powder that hydrates into a hydrogel of an appropriate viscosity after delivery to the desired location. After inhalation, for example, the hydrogel absorbs water to obtain the desired viscosity and then delivers agents to the respiratory system. When administered orally, a hydrogel can be selected that does not absorb water under conditions present in the upper gastrointestinal tract, but which does absorb water under conditions present in the lower gastrointestinal tract (i.e., at a pH greater than about 6.5). Such hydrogels are well known to those of skill in the art. The use of such compositions can optimize the delivery of agents to the lower gastrointestinal tract.

In another embodiment, the nanoparticles are formulated. For example, in a gel, alone or with enhancer, for simultaneous administration. Alternatively, as parts of a kit, for separate administration. The enhancer can be administered simultaneously with or after administration of the composition including the viscous gel and agent to be delivered. The administration schedule (e.g., the interval of time between administering the enhancer and administering the gel composition) can be readily selected by one of skill in the art to maximize receptor expression and/or binding before exposure of the cell surface to the agent to be delivered.

The dosage will be expected to vary depending on several factors, including the patient, the particular bioactive compound to be delivered, and the nature of the condition to be treated, among other factors. One of skill in the art can readily determine an effective amount of the bioactive compound or compounds to administer to a patient in need thereof

III. Applications for the Compositions and Methods

A. Sterilization or killing of reproductive tissue

The compositions described herein can be used to sterilize mammals (animals and humans) and/or treat certain sex hormone related diseases such as cancer of the prostate or cancer of the breast. The preferred targeting molecules for this application are various peptide hormone molecules such as certain analogs of gonadotropin-releasing hormone, GnRH. The preferred agent to be delivered are the cytotoxic agents. The term gonadotropin-releasing hormone will usually be abbreviated as “GnRH ” and as used herein include analogs thereof.

The carriers are specifically targeted to the gonadotropin-secreting cells of the anterior pituitary gland. Indeed they are the only cells to which the gonadotropin-releasing hormone portion of applicant's conjugates will bind. The toxic compounds serve to permanently destroy a subpopulation of the anterior pituitary cells and thereby eliminate the gland's ability to secrete gonadotropins. This direct chemical attack upon the pituitary gland, in turn, causes the animal's gonads to atrophy and lose their ability to function for reproductive purposes. Without functioning gonadotrophs, an animal is not able to secrete luteinizing hormone (LH) and follicle-stimulating hormone (FSH) and thus is rendered sterile. Consequently, these compositions have great potential utility in human medicine as well as in veterinary medicine. This follows from the fact that there are several important biological reasons for employing castration and antifertility drugs in humans. For example, breast and prostate cancers are but two examples of sex steroid-dependent tumors which respond to such hormonal manipulation. At present, the only reliable way to inhibit steroid-dependent tumor growth is through administration of counter-regulatory hormones (e.g., DES in prostate cancer), sex-steroid hormone binding inhibitors (e.g., tamoxifen in breast cancer) or surgical castration. Thus the potential medical uses of such chemical castration compounds are vast and varied. For example, prostate cancer remains an important cause of cancer deaths and represents the second leading cancer of males. It should also be noted that for purposes of disease and/or fertility control, especially in humans, it may be desirable to use the compositions to ablate pituitary gonadotrophs in conjunction with other modes of treatment. For example, it is anticipated that chronic administration of progestins and estrogens to females and androgens to males might be necessary to prevent loss of secondary sex characteristics, behavior and osteoporosis. Another area of application in human medicine is treatment of endometriosis. This condition, which produces painful growth of endometrial tissue in the female peritoneum and pelvis also responds to inhibition of sex steroid synthesis. Those skilled in this art will also appreciate that the herein disclosed compounds could be used to partially reduce sex-steroid secretions, and thus reduce or eliminate certain hormone related behavior problems while retaining improved growth stimulation.

The dose/time adjustments associated with the use of these compounds can vary considerably; however, these compounds are preferably administered by injection into a mammal in concentrations of from about 0.1 to about 10 milligrams per kilogram of the mammal's body weight. Sterilization may be accomplished with as few as one injection; but multiple treatments (e.g., based upon concentrations of from about 0.03 milligrams once every 4 days to about 1 milligram per kilogram of body weight for 20 days) are alternative sterilization schemes. Furthermore, as sterilization agents, the compositions can be used before or after puberty.

The present invention will be further understood by reference to the following non-limiting examples.

The following examples show that toxin could be encapsulated in nanoparticles, and that nanoparticles labeled with a targeting molecule could be targeted preferentially to target cells.

### EXAMPLE 1

### Conjugation of a Nanoparticle with a Targeting Molecule

A system based on LHRH was used to obtain a nanoparticle carrier with a targeted molecule bound to its surface.

Materials and Methods

Reagents. NeutrAvadin™ labeled microspheres (0.04 μm, polystyrene, yellow-green fluorescent 505/515) were purchased from Molecular Probes (Eugene, Oreg.). All other chemicals including [Biotinyl-Gln¹]-LHRH were obtained from Sigma (St Louis, Mo.).

The following procedure was used to prepare the nanoparticle-LHRH complex, with all steps performed at room temperature:
1) 1 mg of [Biotinyl-Gln¹]-LHRH was dissolved in 5 mL of Dulbecco's Phosphate Buffered Saline (DPBS) without Mg⁺ and Ca²⁺ ions to create a stock solution.
2) The NeutrAvadin™ labeled microspheres were sonicated at 100 g for 10 minutes before undergoing dialysis for 5 hours to remove any possible chemical preservatives. The dialysis buffer was a solution of 1 part DPBS, 1 part distilled water.
3) To 0.4 mL of the NeutrAvadin™ labeled microspheres was added 218 mL of B-LHRH stock solution and 218 mL of a 1% solution of BSA in DPBS.
4) Dialysis was performed over a 24-hour period to remove any unbound reagents, using the same buffer as in part (2). The buffer was exchanged every 4 hours.

The clarity of the resulting solution suggested that particle-particle aggregation, a notorious problem with targeted nanoparticles, did not occur, i.e. that the nanoparticles remained in solution at a submicron size.

### EXAMPLE 2

### Targeting of Nanoparticle Complex to Selected Cells

The following showed that nanoparticles labeled with LHRH could be targeted to cells expressing LHRH receptor.

Materials and Methods

Reagents. Alpha minimum essential medium was purchased from JRH Biosciences (Lenexa, Kans.). Penicillin-Streptomycin solution was obtained from ATCC (Manassas, Va.). All other chemicals were purchased from Sigma (St Louis, Mo.).

Cell Culture and Preparation. Rattus norvegicus (rat) anterior pituitary cells, designation RC-4B/C, were purchased from ATCC (Manassas, Va.). The cells were incubated in a 37° C., 5% CO₂ atmosphere in a medium comprising Dulbecco's modified Eagle's medium containing 2.2 g/L sodium bicarbonate, 4 mM L-glutamine, and 4.5 g/L glucose, 45%; alpha minimum essential medium with 1 g/L glucose, 45%; supplemented with 15 mM HEPES, 0.2 mg/mL bovine serum albumin, 2.5 ng/ml epidermal growth factor; dialyzed heat-inactivated* fetal bovine serum, 10%. The medium was supplemented with 50 units/mL penicillin and 0.05 mg/mL streptomycin. *FBS was heat-inactivated using the ATCC-recommended standard procedure.

Following ATCC guidelines, the complete growth medium was refreshed every 2-3 days and subculture was performed every 5-6 days. The following procedure was used:
1) All medium was discarded and a 37° C. solution of 0.05% trypsin, 0.02% EDTA, 0.1% glucose was added to the cell culture vessel at a volume of 0.1 mL per cm² of cell coverage.
2) The cells were kept at 37° C. for 7-10 minutes, during which time they detached from the surface of the culture vessel.
3) An equal volume of complete growth medium was added to the culture vessel before the cell suspension was transferred to a sterile tube and pelleted through centrifugation at 100 g for 10 minutes.
4) The pellet was resuspended in fresh 37° C. growth medium and placed into sterile culture vessels such that a subculture ratio of 1:2 or 1:3 was achieved.

Endocytosis.

Experiments then were used to establish that targeted cells can internalize a nanoparticle-targeting molecule conjugate with greater efficiency than they internalize an unconjugated nanoparticle of the same physical properties. The experimental groups were as follows: The test nanoparticle/targeting molecule complex was a conjugate of NeutrAvadin™ labeled microspheres with [Biotinyl-Gln¹]-LHRH, prepared as described in Example 1 above. The positive control group comprised carboxylate-modified microspheres (0.04 μm, polystyrene, yellow-green fluorescent 505/515) obtained from Molecular Probes (Eugene, Oreg.). The negative control group comprised cells to which no particles had been added; this group was used to determine cell autofluorescence. After incubating equal concentrations of conjugate or carboxylated nanoparticles with separate volumes of cell suspension (see procedure below), the amount of fluorescence associated with the cells was recorded using a flow cytometer.

Procedure

The procedure was identical for all three experimental groups as described above (test, positive control, negative control). Henceforth groups 1 and 2 are referred to as ‘particles’ 14 hours in advance of flow cytometry:
1) Cells were trypsinized and centrifuged according to the subculturing procedure described above.
2) Each pellet was resuspended in 2 mL preheated calcium-free medium of the following composition: minimum essential medium, Spinner Modification, containing 1 g/L glucose, 100%; supplemented with 15 mM HEPES and 0.2 mg/mL bovine serum albumin
3) Cell suspension underwent constant gentle mixing at room temperature for 12 hours.

2 hours prior to flow cytometry:
4) Cells were heated to 37.0° C. (in the event of overnight sedimentation, gentle resuspension was first performed)
5) After sonication (specification of sonication as described in Example 1), particles were incubated with cells at a concentration on the order of 100 particles/cell.
6) After rapid agitation to ensure thorough mixing, the cell suspensions were left to incubate with the particles at 37.0° C. for 10 minutes.
7) The cells were rapidly cooled before centrifugation and resuspension of the resulting pellet (as described in subculturing procedure) in 1 mL of calcium-free medium at 4° C.
8) Propidium Iodide was added to the cell suspensions at a concentration of 50 μl/mL
9) The cells were kept at 4° C. for a further 45 minutes prior to performing flow cytometry.

Results

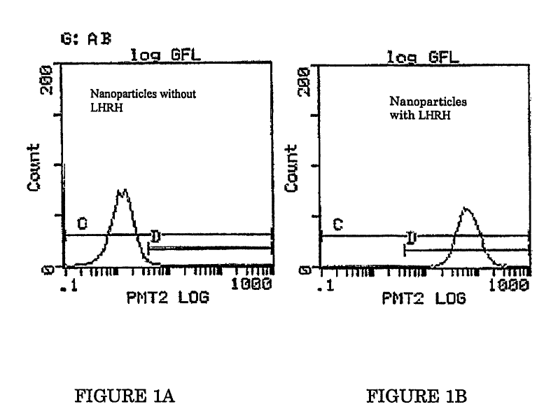

FIGS. 1A and 1B illustrate that the amount of fluorescence associated with cells that had been incubated with the test conjugate was significantly greater than that associated with cells which had been incubated with the control particles. This result supports the claim that a nanoparticle conjugated with a targeting molecule undergoes receptor-mediated endocytosis at a greater rate than does a bare nanoparticle.

### EXAMPLE 3

### Synthesis of a Nanoparticle System Containing a Toxin Payload

Similar nanoparticles can be made containing toxins using the procedures described below:

A nanoparticle system carrying a toxin payload can be made using the procedure as described in: Pautot, Frisken, Weitz “Production of unilamellar vesicles using an inverted emulsion” (submitted for publication). Using Pautot et al's technique, streptavidin-coated lipids (DPPC, DSPC, and similar lipids) can be used to manufacture liposomes. Moreover, by adapting Needham's drug encapsulation technique (Needham D, Newhirst M W. The development and testing of a new temperature-sensitive drug delivery system for the treatment of solid tumors. Advanced Drug Delivery Reviews, 53 (3): 285-305 Dec. 31 2001), it is also possible to load these vesicles with doxorubicin, a model small-molecule toxin currently used in cancer therapy.

The liposomes can be prepared by exposing chloroformic solution of various lipid mixtures to high vacuum and subsequently hydrating the resulting lipid films (DSPC/CHOL) with pH 4 buffers, and extruding them through polycarbonated filters, after a freezing and thawing procedure. It is possible to use DPPC supplemented with DSPC or cholesterol to increase encapsulation efficiency or increase stability, etc. A transmembrane pH gradient is created by adjusting the pH of the extravesicular medium to 7.5 by addition of an alkalinization agent. The anticancer drug doxorubicin is subsequently entrapped by addition of the drug solution in small aliquots to the vesicle solution, at an elevated temperature, to allow drug accumulation inside the liposomes.

Once such streptavidin-coated and doxorubicin-loaded liposomes are made, the procedure described in example 1 can be repeated in order to attach [Biotinyl-Gln¹]-LHRH to the liposomes forming a liposome-LHRH complex. The protocol outlined in example 2 can then be followed to study the efficacy of toxin delivery to Rattus norvegicus anterior pituitary cells.

1. A composition for delivery of a therapeutic, prophylactic or diagnostic agent comprising:
a nanoparticulate carrier comprising therapeutic, prophylactic or diagnostic agent to be delivered, and
a targeting molecule bound to the surface of the carrier,
wherein the carrier is targeted to a specific population of cells where the agent is to be delivered.

2. The composition of claim 1 wherein the carrier is selected from the group consisting of liposomes and microparticles.

3. The composition of claim 2 wherein the microparticles are formed of the agent to be delivered.

4. The composition of claim 2 wherein the microparticles are formed of a polymeric material.

5. The composition of claim 1 wherein the agent to be delivered is a cytotoxic compound.

6. The composition of claim 1 wherein the targeting molecule is a hormone.

7. The composition of claim 1 further comprising a gel enhancing uptake of the carrier into a cell.

8. A method of delivering a therapeutic, prophylactic or diagnostic agent comprising administering to a patient or tissue a composition comprising a nanoparticulate carrier comprising therapeutic, prophylactic or diagnostic agent to be delivered, and
a targeting molecule bound to the surface of the carrier,
wherein the carrier is targeted to a specific population of cells where the agent is to be delivered.

9. The method of claim 8 wherein the composition is administered to sterilize an animal.

10. The method of claim 8 wherein the composition is administered to treat cancer or endometriosis.

11. The method of claim 8 wherein the composition is administered by injection.

12. The method of claim 8 wherein the composition is administered by pulmonary or intranasal or intravaginal or intrarectal delivery.
